(19) United States
(12) Patent Application Publication
(10) Pub. No.: US 2003/0200033 A1
(43) Pub. Date: Oct. 23, 2003

(54) High-throughput alignment methods for extension and discovery

(76) Inventors: Jonathan Segal, Newtonville, MA (US); Hui Huang, Newton, MA (US)

Correspondence Address: Nina L. Pearlmutter, Genome Therapeutics Corporation, 100 beaver Street, Waltham, MA, 02453, US

(21) Appl. No.: 10/123,085

(22) Filed: Apr. 12, 2002

(51) Int. Cl. G06F 19/00; G01N 33/48; G01N 33/50

(52) U.S. Cl. 702/20

(57) ABSTRACT

The invention provides an automated method of simultaneously identifying sequence information extending a plurality of seed sequences. The method consists of: (a) searching a plurality of target sequences with a multiplex query comprising a plurality of seed sequences; (b) identifying a plurality of target sequences substantially aligning with a plurality of seed sequences; (c) selecting a plurality of substantially aligned target sequences containing sequence extending information for a plurality of seed sequences, and (d) repeating steps (a) through (c) using the selected plurality of substantially aligned target sequences as a plurality of seed sequences. Also provided is an automated method of simultaneous identifying a plurality of gene sequences within a plurality of genomic region sequences. The method consists of: (a) pruning nucleic acid sequence elements from a plurality of genomic region sequences to produce a plurality of genomic seed sequences; (b) searching a plurality of target gene sequences with a multiplex query comprising a plurality of genomic seed sequences; (c) identifying a plurality of target gene sequences substantially aligning with a plurality of genomic seed sequences, and (d) locating regions of substantial alignment of the identified plurality of target gene sequences within the plurality of genomic region sequences, the regions of substantial alignment identifying a plurality of gene sequences.

### BACKGROUND OF THE INVENTION

This invention relates generally to genomics and related bioinformatic methods for processing large amounts of nucleic acid sequence information and, more specifically to methods of simultaneously mining large amounts of nucleic acid sequence data for extension and discovery of new sequence information.

The human genome project has resulted in the generation of enormous amounts of DNA sequence information. The generation of this information and achievement of the complete sequencing of the human genome has required numerous technical advances both in sample preparation and sequencing methods as well as in data acquisition, processing and analysis. During the project's quick evolution, it has brought to fruition the scientific fields of genomics, proteomics and bioinformatics. As a result, a complete draft sequence of the human genome was published in February of 2001. Moreover, in developing and improving processes for sequencing, processing and analysis of genomic quantities of sequence information, the complete genome sequences for numerous procaryotic organisms and for at least two different eucaryotic organisms have now been reported with several others approaching completion.

Automated DNA sequencing procedures have been developed that require essentially little to no human intervention outside of sample preparation. For example, computerized robotics generate and perform sequencing reactions and the resulting signals are detected by sensors which are read into a computer. Algorithms and software are available which analyze and process signal from noise in order to detect the nucleotide sequence for a corresponding reaction. The signals can then be transformed into a graphical display or other readout formats convenient for the user.

The number and rate of different reactions which can be performed currently exceeds hundreds of thousands of bases (b) per day. Analyzing and processing such information into useful strings that reflect the nucleotide sequence of the genes and chromosomes from which they were derived can be performed by assembly or alignment algorithms and their corresponding computer executable code. Such programs compare and organize a multiplicity of like sequences into groups and merge them into a single contiguous sting of nucleotides representing the sequence of a DNA strand.

Advancements in automated sequencing procedures and the genomic era emphasis on data acquisition has resulted in the accumulation of a vast amount of sequence data. However, the ability to meaningly organize, analyze and interpret archives of sequence information into structural relationships or into biologically relevant contexts has been lagging. For example, genomic sequence databases contain an enormous content of gene and genomic sequence information. However, only a small portion of such databases constitute unique sequence information due to deposits of redundant and overlapping sequence information. Data analysis, data management and lack of efficient curating procedures all contribute to the current information state of sequence databases. Out of the many databases that have been developed, to date there are only a few genomic databases that contain non-redundant gene sequence information.

A similar situation has occurred with sequence databases other than genomic databases. For example, the influence of automation and emphasis on data acquisition in the genomic era also lead to the development of several expressed sequence tags (ESTs) databases. Such databases are essentially the result of high-throughput sequencing and deposit of cDNA sequence information with little to no analysis or curing of the raw data. Because such sequences represent the expressed portion of a genome, a cross-reference of this sequence data to genomic sequence information should lead to the identification of structural gene regions and their distinction from intragenic or other genomic region sequence. However, EST databases are fraught with the same drawbacks as genomic databases in that there is a plethora of redundant and overlapping sequence data with essentially no meaningful organization or curating. This problem is further complicated by the magnitude of new sequence information being generated. For example, it is estimated that as many as 6,000 to 8,000 new EST sequences are deposited every day.

Regardless of the problems with size and redundancy of the various nucleic acid sequence databases, they are still valuable sources of information for genetic discovery and analysis. The challenge continues to be how to tap into such enormous amounts of information, extract and use only the meaningful portion to address a particular problem or to extend the useful set of meaningful sequence information.

Thus, there exists a need for computational methods and repertoires that can efficiently analyze, determine and organize large amounts of sequencing data into meaningful structural and biologically relevant relationships. The present invention satisfies this need and provides related advantages as well.

### SUMMARY OF THE INVENTION

The invention provides an automated method of simultaneously identifying sequence information extending a plurality of seed sequences. The method consists of: (a) searching a plurality of target sequences with a multiplex query comprising a plurality of seed sequences; (b) identifying a plurality of target sequences substantially aligning with a plurality of seed sequences; (c) selecting a plurality of substantially aligned target sequences containing sequence extending information for a plurality of seed sequences, and (d) repeating steps (a) through (c) using the selected plurality of substantially aligned target sequences as a plurality of seed sequences. Also provided is an automated method of simultaneous identifying a plurality of gene sequences within a plurality of genomic region sequences. The method consists of: (a) pruning nucleic acid sequence elements from a plurality of genomic region sequences to produce a plurality of genomic seed sequences; (b) searching a plurality of target gene sequences with a multiplex query comprising a plurality of genomic seed sequences; (c) identifying a plurality of target gene sequences substantially aligning with a plurality of genomic seed sequences, and (d) locating regions of substantial alignment of the identified plurality of target gene sequences within the plurality of genomic region sequences, the regions of substantial alignment identifying a plurality of gene sequences.

### BRIEF DESCRIPTION OF THE DRAWINGS

FIG. 1 shows a flow chart of sequence extension analysis that includes pruning and computational load sharing.

FIG. 2 shows a schematic of a nucleic acid extension process using EST seed and target sequences.

FIG. 3 shows a flow chart of gene discovery analysis that includes pruning and computational load sharing.

FIG. 4 shows a graphic view of an extension analysis resulting from a seed sequence.

### DETAILED DESCRIPTION OF THE INVENTION

This invention is directed to automated methods for gene extension and discovery. The computational methods of the invention enable the simultaneous search and identification of large numbers of similar nucleic acid sequences within an enormous number of diverse and different sequences. The ability to rapidly search and identify related sequences within nucleic acid sequence databases allows the matching of deposited sequence information to known nucleic acids for the extension, by assignment, of the known sequence with non-overlapping portions of sequence information in the newly discovered matching sequence. By the same approach, as yet undiscovered genes within the repertoire of genomic sequence databases similarly can be identified and extended using the methods of the invention. One advantage of the methods of the invention is that they distribute the computational effort over available computing resources through the use of a multiplex system of search and analysis. The automated methods of the invention reduce a plurality of different sequences into single data elements or search queries for sequence analysis procedures while outputting the non-multiplexed forms of each sequence. The automated methods of the invention also employ a triage procedure to cull out undesirable sequence information, which allows computational resources to focus on the only the relevant sequences within a data set of nucleic acid sequence information.

As used herein, the term “sequence extending information” is intended to mean nucleic or amino acid sequence information that, upon combining with a reference sequence, provides additional primary nucleotide or amino acid sequence to the reference sequence. Sequence extending information can be, for example, the determination of new primary sequence for a reference sequence or the identification of a new association of a known primary sequences with a reference sequence. The additional primary nucleotide or amino acid sequence merges with the reference sequence so as to expand the primary nucleotide or amino acid sequence by the amount of newly determined or identified sequence information. Extension of sequence information can be, for example, at either 5′ or 3′ termini of a nucleic acid reference sequence or at either amino (N) or carboxyl (C) termini of an amino acid reference sequence or within an internal region of the reference sequence. A specific example of obtaining sequence information that extends a terminus of a reference fragment includes obtaining a nucleic or amino acid fragment that partially overlaps, by sequence alignment, with a terminal region of the reference sequence. The non-overlapping portion of the fragment constitutes nucleic acid sequence extending information. A specific example of sequence information extending an internal region of a reference sequence includes a nucleic or amino acid fragment that overlaps with a reference sequence at two non-contiguous regions with a non-overlapping intervening portion. Similarly, the non-overlapping intervening portion of the fragment constitutes nucleic or amino acid sequence extending information. Sequence extending information corresponding to internal regions include, for example, introns, splice junctions, domain swapping and the like. Various other examples of nucleic or amino acid sequence extending information well known to those skilled in the art also exist and are included within the meaning of the term.

As used herein, the term “plurality” is intended to mean two or more different referenced molecules or sequences. Therefore, a plurality constitutes a population of two or more different members. Pluralities can range in size from small, to large, to very large. The size of small pluralities can range, for example, from a few members to tens of members. Large pluralities can range, for example from about 100 members to hundreds of members. Similarly, very large pluralities can range from about 1000 members, to thousands, tens of thousands, hundreds of thousands and greater than one million members. Therefore, a plurality can range in size from two to well over one million members as well as all sizes, as measured by the number of members, in between. Accordingly, the definition of the term is intended to include all integer values greater than two. A upper limit of a plurality of the invention is limited only by the available computational power.

As used herein, the term “seed” or “seed sequence” is intended to mean a reference sequence that is to be extended. When used in reference to a nucleic acid sequence, the reference sequence will be extended by the addition or incorporation of nucleic acid sequence extending information. Similarly, when used in reference to an amino acid sequence, the reference sequence will be extended by the addition or incorporation of amino acid sequence extending information. A seed sequence of the invention can constitute any form of nucleic or amino acid sequence for which the user desires to obtain unrecognized primary nucleotide or amino acid sequence information. Such forms of nucleic acid sequences can include, for example, genomic sequence, gene sequence, such as gene structural regions, and expressed sequences such as expressed sequence tags (ESTs) and copied messenger RNA (cDNA). Any of the above forms of nucleic acid sequences can be obtained from, for example, sequence databases or directly from read sequence data which is produced de novo. Forms of amino acid sequences can include, peptide, polypeptide, protein, or any of the above forms of coding region nucleic acid translated into primary amino acid sequence. Similarly, such forms of sequence can be obtained from sequence databases, proteomic databases or from raw data. The unrecognized primary sequence can include, for example, adjacent, flanking or internal primary nucleotide or amino acid sequence present in a larger nucleic acid, a larger polypeptide, or component fragments thereof, but unrepresented in the available form of the reference sequence. Such adjacent, flanking or internal sequence information generally can be, for example, contiguous with a seed sequence termini, an internal boundary or with sequence extending information of the seed sequence. Therefore, a seed sequence constitutes a fragment or portion of a larger nucleic or amino acid acid sequence, whether represented as a single sequence or multiple component fragment sequences, for which the association or identification is to be made.

As all naturally occurring nucleic acids derive from genomic nucleic acid, a reference to a specific type of nucleic acid sequence is intended to refer to a subcategory of a genomic nucleic sequence. Similarly, and unless specifically referred to otherwise, the use of the general term “nucleic acid” without reference to genomic or a subcategory thereof of genetic information is intended to include both naturally occurring and non-naturally occurring nucleic acids or nucleic acid sequence. For example, the term “genomic,” as used herein, refers to a nucleic acid or nucleic acid sequence that corresponds to a region of a chromosome. Genomic sequences can contain, for example, genetic structural regions, such as a gene, including exons, introns or other substructures thereof, intragenic region sequence, centromeric region sequence, or telomeric region sequence, as well as other chromosomal regions well known to those skilled in the art. The term “gene” as used herein refers to a chromosomal region encompassing the genetic structural elements of a gene, or a fragment thereof.

Similarly, as all naturally occurring peptides, polypeptides and proteins derive from coding region nucleic acid sequence, a reference to a specific type of coding region nucleic acid sequence also is intended to refer to its translated amino acid sequence. Similarly, and unless specifically referred to otherwise, the use of the general terms “amino acid sequence” or “polypeptide” is intended to include both naturally occurring and non-naturally occurring polypeptides or amino acid sequences. It also is intended to be understood that the automated methods of the invention can be employed equally with any polymer sequence composed of monomer building blocks because the algorithms, methods and processes described herein search, manipulate, analyze and process character strings. Such polymers include, for example, organic polymers and macromolecules with monomer building blocks such as nucleic acid, polypeptide, carbohydrate and the like.

Because the algorithms and corresponding automated methods are equally applicable to searching all types of monomer-composed polymer sequences, those skilled in the art will understand that where a polymer is encoded by another type of sequence, one can implement the methods of the invention in search routines employing either its encoded form, translated from or reverse-translated form. For example, sequence extension or discovery can be performed on a nucleic acid sequence in nucleic acid computational space or it can be translated into amino acid sequence and performed in polypeptide computational space. The former will yield nucleic acid sequence extending information and the latter will yield amino acid sequence extending information. Similarly, for example, an amino acid sequence can be searched directly in polypeptide computational space to yield amino acid sequence extending information, or alternatively, it can be reverse translated into its coding nucleic acid sequence and searched in nucleic acid computational space to yield nucleic acid sequence extending information. Therefore, the sequence extension and discovery methods of the invention also are applicable for sequence analysis in translated or reverse translated computational search space.

Accordingly, nucleic acid seed sequences and, as described further below, nucleic acid target sequences can be any and all categorical types of nucleic acids, ranging from genomic to non-naturally occurring nucleic acid sequences. Similarly, amino acid seed sequences and amino acid target sequences also can be any category of peptide, polypeptide or protein as well as correspond to any of the categorical types of nucleic acids described herein that contain coding region sequence or an open reading frame (ORF). With reference to nucleic acid sequences, for example, a genomic seed sequence refers to a reference nucleic acid sequence which is to be extended that is derived from a genomic nucleic acid. Similarly, a gene seed sequence refers to, for example, a reference nucleic acid sequence corresponding to a gene or a fragment thereof. As there are numerous forms and nucleic acid products from a gene, a gene seed sequence or a target gene sequence can include sequences derived from or corresponding to these various forms. For example, a seed or target sequence can derived from, or correspond to, a gene, a cDNA, an an EST, hnRNA and RNA since all of these types of nucleic acids represent or contain sequence information corresponding to their encoding gene. It is understood to those skilled in the art that the structural portion of a gene includes both coding and non-coding regions a gene.

As used herein, the term “target” or “target sequence” is intended to mean a sample sequence that is probed for containing sequence extending information. When used in reference to a nucleic acid sequence, a sample sequence that partially overlaps with a nucleic acid seed sequence will contain, as the non-overlapping portion, nucleic acid sequence extending information. When used in reference to an amino acid sequence, a sample sequence that partially overlaps with an amino acid seed sequence will contain, as the non-overlapping portion, amino acid sequence extending information. The non-overlapping portion sequence of a target sequence includes, for example, the unrecognized primary sequence information of its cognate seed sequence. As with nucleic and amino acid seed sequences, a nucleic or amino acid target sequence of the invention can constitute any form of nucleic or amino acid sequence for which the user desires to probe for unrecognized primary nucleotide sequence information or primary amino acid sequence information. Such forms of nucleic acid sequences can include, for example, genomic sequence, gene sequence, such as gene structural regions, and expressed sequences such as expressed sequence tags (ESTs) and copied messenger RNA (cDNA). Such forms of amino acid sequences can include, for example, peptide, polypeptide, protein or amino acid sequence corresponding to nucleic acid coding region sequence or ORF sequence.

As used herein, the term “prune” or “pruning” is intended to mean reducing or eliminating referenced subject matter. The term is therefore intended to mean that the referenced nucleic acid sequence information or amino acid sequence information is, in part or in whole, removed or ignored in the methods of the invention. Pruning can be accomplished using various computational methods well known to those skilled in the art. Such methods include, for example, deletion, omission, filtering, masking, and selection so long as execution of such instructions results in a reduction or elimination of sequence information having attributes specified for removal. Additionally, pruning also can be performed by partial or completely manual methods, including, for example, human intervention.

As used herein, the term “superfluous” when used in reference to nucleic acid sequence information or amino acid sequence information, is intended to mean sequence information that is dispensable or nonessential for executing one or more steps in the methods of the invention. The term therefore is intended to include nucleic or amino acid sequence information that is unnecessary, unneeded or unwanted for executing one or more steps in the methods of the invention. One measure of superfluous nucleic or amino acid sequence information is redundancy. Redundant nucleic acid or amino acid sequences include, for example, identical or inclusive sequences and repetitive elements. Another measure of superfluous nucleic acid or amino acid sequence information is non-relevancy. Non-relevant sequences include, for example, those which fail to align with a seed sequence cluster, which includes overlapping cognate target sequence, and those which contain sequence artifacts. Other measures of superfluous nucleic or amino acid sequence information are well known to those skilled in the art and also can be employed in the methods of the invention given the teachings and guidance provided herein. Therefore, superfluous nucleic acid or amino acid sequence information can include, for example, non-overlapping target sequences and target sequences that are substantially inclusive with a seed sequence cluster.

As used herein, the terms “align,” “alignment” or grammatical forms thereof, when used in reference to a comparison of nucleic acid or amino acid sequences is intended to mean a representation of two or more sequences sharing matches, mismatches or gaps at each nucleotide or amino acid position when placed in proper relative position or orientation. The degree to which positions match or correctly align is a measure of their sequence similarity. Sequences that completely match, without mismatches or gaps, are considered identical. In contrast, sequences that do not align, or exhibit a frequency of matching positions expected to occur by chance, are considered non-identical. Sequences that align with match frequencies greater than chance are considered significant and fall within the meaning of the term as used herein. Therefore, the term “substantial” as used herein with reference to the degree of nucleic acid or amino acid sequence alignment is intended to mean that the compared sequences are the same, or are deemed to be the same, given for example, the sequencing error rate inherent in input data, the algorithm used for comparison and the search and alignment parameters employed in a particular run analysis. Given a particular computational background and sequencing data source, those skilled in the art will know, or can determine, a range or boundary of nucleotide or amino acid match that is acceptable for deeming two sequences to be the same.

Methods for aligning two or more nucleic acid or amino acid sequences are well known in the art. Such methods include, for example, local sequence alignment, pairwise alignment and multiple alignment. Similarly, alignment algorithms and written instructions their automated implementation are similarly well known to those skilled in the art. Such algorithms and instructions include, for example, dynamic programming, heuristic algorithms, linear space, hidden Markov models (HMM), Barton-Sternberg algorithm, profile HMMs, Feng-Doolittle progressive alignment, multidimensional dynamic programming, Smith-Waterman algorithm, Neddle and Wunsch algorithm, BLAST, FASTA, d2_cluster, Phrap, and CLUSTAL. Any of these methods, as well as others well known to those skilled in the art can be used in the automated methods of the invention.

As used herein, the term “consensus” is intended to mean the reduction of a nucleotide or amino acid position in a multiple alignment to a single inclusive base or residue character. The single inclusive base or residue can represent, for example, a nucleotide or residue occurring at the referenced position that occurs most frequently or is the most likely to occur based on quality scores or error models. Inclusive positions also can include, for example, two or more alternatives at a particular position where the alternatives are equally likely to occur. An example of an inclusive consensus nucleotide sequence and its corresponding nomenclature is shown below with reference to FASTA format files. Consensus sequences can be generated by, for example, alignment, assembly or other relative comparison of a plurality of nucleic acid or amino acid sequences and frequency determination at some or all positions of interest.

As used herein, the term “cluster” or “sequence cluster” is intended to mean an organization of sequences as groups. Groups specified by a clusters can have, for example, attributes selected by the user or predetermined by the analysis parameters. For example, when referring to substantially aligned nucleic or amino acid sequences, a cluster of such sequences is intended to mean the collection of nucleic or amino acid sequences that have some region of sequence similarity that is the same, or deemed to be the same, between each member within the group. Therefore, “clustering” refers to the process of selecting or identifying individual nucleic acid or amino acid sequences as a member of a specified group.

As used herein, the term “automated” or “automated process” is intended to mean a self-controlled operation of an apparatus, process or system by mechanical or electrical devices, or both, that can substitute for human intervention, including cognitive decision processes. Minor human interventions which do not substantially affect the primary functions of the process are included within the definition of the term. Such minor interventions can include, for example, input and export of data, including beginning and ending data, as well as viewing and user analysis of intermediate or final output data. Generally, a process is automated through the control of a computer, which is a programmable electronic device that can store, retrieve and process data. An algorithm refers a series of procedural instructions that define the automated steps of a method. In a computerized process, the algorithm defines a list of coded instructions implemented by the computer.

In large scale nucleic acid sequencing projects or proteomic projects, immense amounts of sequence information can be generated in very short periods of time. Computer automated processes have been employed to generate and process such quantities of information within usable time frames. The accurate analysis and meaningful organization of the information becomes important because the identification of full-length genes or encoded polypeptides, complete coding regions or the discovery of unrecognized genes within a genomic sequence region can dramatically impact the understanding of physiological processes as well as the diagnosis and therapeutic intervention of diseases. Therefore, the beneficial effect of genome and proteomic sequence information to the health care industry will correlate with the attainment of accurate and organized information that reveals biologically relevant sequence content. The methods of the invention are useful in efficiently identifying and assimilating large numbers of diverse sequences into relevant biological contexts. Such methods are useful in simple and complex systems which generate, process and analyze both small numbers of sequences as well as large numbers, including hundreds of thousands of sequences.

The invention provides an automated method of simultaneously identifying sequence information extending a plurality of seed sequences. The method consists of (a) searching a plurality of target sequences with a multiplex query comprising a plurality of seed sequences; (b) identifying a plurality of target sequences substantially aligning with a plurality of seed sequences; (c) selecting a plurality of substantially aligned target sequences containing sequence extending information for a plurality of seed sequences, and (d) repeating steps (a) through (c) using the selected plurality of substantially aligned target sequences as a plurality of seed sequences.

The automated methods of the invention provide an algorithm that can be implemented by a computer for the identification of nucleic acid or amino acid sequence extending information. The algorithm, and its corresponding computer implemented code, advantageously combine computational search, alignment and clustering processes to overcome prohibitively slow semi-manual processes that are labor intensive or brute-force computational approaches. For example, the automated methods of the invention are about 10- to 100-fold faster tan searching a comparable number of seed sequences against the unique gene cluster database UniGene and about 1000-fold or greater than searching a single seed sequence at a time.

Nucleic acid or amino acid sequence extending information refers to a nucleic acid or amino acid sequence that increases or adds new primary sequence to a reference nucleic acid or amino acid sequence. In its non-computational form, nucleic or amino acid sequence extending information can be generated by, for example, step-wise sequencing of an adjacent region of a reference nucleic acid sequence template or polypeptide. The nucleic acid process is step-wise because it proceeds by obtaining a reference nucleic acid sequence, generating a primer and then extending the primer into the adjacent region to generate new sequence. Similarly, the amino acid process is step-wise because it involves the repetitive iteration of sequencing one residue at a time. The newly sequenced portion adds sequence to the reference sequence terminus to extend the known primary sequence of the reference nucleic acid.

In it computational form, the process proceeds simultaneously and there is no requirement for prior sequence knowledge or need to actually sequence an adjacent region of the reference sequence Instead, the methods of the invention take for granted that an adjacent region sequence has been deposited somewhere within the vast repertoire of sequence databases. The extension process of the invention follows a walking procedure where new sequence information is generated through the identification of non-coterminus, overlapping sequences. Overlapping sequences indicate that the compared sequences derive from the same genomic sequence or gene, or from the same polypeptide, and as such, that they are two fragments of the same, larger nucleic acid or polypeptide. The non-coterminus nature of selected fragment indicates that at least one of the compared sequences will contain sequence information different from, and additional to the internally terminated sequence. The extension process can be with or without prior knowledge of either the initiating seed sequence or the extending target sequence because such sequences will be contained within a database and therefore in existence.

Because nucleic acids encode biological information in a double-stranded, complementary form or in single-stranded forms corresponding to either a sense or a complementary anti-sense strand, those skilled in the art will understand that references herein to a nucleic acid or nucleic acid sequence of the invention describes either or both strands of a nucleic acid molecule. Therefore, two sequences can be overlapping and for that reason be complementary, for example, with respect to sense strands, anti-sense strands, complementary strands, or both as it is well known to those skilled in the art that knowledge of a single strand of nucleic acid sequence necessarily provides the complementary strand. Algorithms and automated processes are similarly well known in the art that can, for example, search, align, assemble, cluster, compare and manipulate either or both the sense and complementary strand of nucleic acid sequences. Such algorithms and automated processes similarly, for example, search, align, assemble, cluster, compare and manipulate amino acid sequences in like manner. Thus, reference to a nucleic acid or amino acid reference sequence, seed sequence or target sequence includes a description of both its sense and complementary sequence and its translated amino acid sequence.

Sequence extending information includes all forms of newly identified sequence information because such information will increase the amount of primary sequence for a reference sequence. The extended sequence information can be, for example, adjacent or contiguous with a terminus of a reference sequence, or internal to the reference sequences termini. Adjacent sequence extending information can be, for example, newly identified primary sequence that is 5′ or 3′ to a nucleic acid reference sequence terminus or N- or C-terminal to an amino acid reference sequence terminus. Contiguous sequence extending information can be, for example, newly identified sequence that begins immediately 5′ or 3′ to a reference nucleic acid sequence terminus or immediately N- or C-terminal to a reference amino acid sequence terminus. Sequence extending information that is internal to a reference sequence terminus or termini can be, for example, the identification of an intron's primary nucleic acid sequence or of an duplicated domain primary amino acid sequence. In all such cases, the acquisition of adjacent, contiguous or internal primary sequence information will nevertheless increase the amount of primary sequence for a reference sequence.

As described previously, the automated methods of the invention can be practiced with nucleic acid sequence, amino acid sequence, or other polymer sequence alike. The algorithms and computational processes can be implemented on monomer-composed primary sequence of a polymers because such monomer building blocks can be represented and manipulated in character, string or word form and formats during the computational processes of the invention. Accordingly, the invention will be described below by reference to nucleic acids and nucleic acid sequences. However, given the teachings and guidance provided herein, those skilled in the art will known that the same or similar process can be implemented in ordinary course of procedure with polypeptides and polypeptide sequences as well as with other monomer-composed polymers and their corresponding primary sequences. For example, to implement the automated methods of the invention with amino acid sequence information, those skilled in the art will know to search an amino acid sequence database with a query of amino acid seed sequences. Implementation is therefore a matter of searching the desired computational space. Thus, the description below with reference to nucleic acids and nucleic acid sequences is nintended to be exemplary.

A seed sequence constitutes a reference sequence for which additional primary nucleic acid sequence is desired. Seed sequences can be, for example, any form of nucleic acid sequence that sequence extending information is to be obtained. For example, seed sequences can constitute a genomic sequence, such as a genomic region or a gene, or fragments thereof, as well as expressed sequences such as cDNA and ESTs, or fragments thereof. The type of seed sequences to employ in the methods of the invention will depend on the design of the user and the objective to be obtained. For example, a user can achieve the extension of reed sequence, coding sequence region of a gene or an open reading frame (ORF) using a cDNA or EST seed sequence. Similarly, a gene can be extended using, for example, a genomic region sequence, a gene sequence or a reed sequence as a seed sequence. Various other forms of such seed sequences as well as others well known to those skilled in the art, including nucleic acid fragments, exons and introns, for example, can similarly be used in the methods of the invention to obtain sequence extending information.

A target sequence constitutes a nucleic acid sequence that is to be searched for nucleic acid sequence extending information. Those target sequences identified as having non-coterminus, overlapping nucleic acid sequences compared to a nucleic acid seed sequence will contain sequence extending information for that input seed sequence. As with seed sequences, a target sequence also can be, for example, any form of nucleic acid sequence that sequence extending information is to be produced. For example, target sequences can constitute genomic sequences, genes, cDNA, ESTs, and read sequences, or fragments thereof. Similarly, the type of target sequences to employ in the methods of the invention will depend on the design of the user and the objective to be obtained. For example, a user can achieve the extension of coding sequence region of a gene or an open reading frame (ORF) using cDNA or EST target sequences. Similarly, a gene can be extended using, for example, any of either genomic region sequences, gene sequences, cDNA sequences, ORF sequences or EST sequences as target sequences. Various other forms of such target sequences as well as others well known to those skilled in the art, including nucleic acid fragments, exons and introns, for example, can similarly be used in the methods of the invention to produce sequence extending information.

Given the teachings and guidance provided herein, those skilled in the art will understand that more specific nucleic acid sequence extending information will be obtained with more refined pairings between seed and target sequence searches. For example, searching genomic target sequences with a query of genomic seed sequences can result in the identification of sequence extending information that can include, for example, all genetic structural region sequences. This result can be obtained because genomic regions are inclusive of all genetic structural regions and therefore can contain genes, introns, exons, intragenic region sequence and the like, and because both seed and target sequences similarly can contain a full range of all regions and elements. However, when one sequence category is refined compared to the other, in that it contains a fewer number of possible genetic structural regions or sequence elements relative to its search partner, the sequence extending information produced will be specific to the refined category of either the seed or target sequence.

For example, searching a genomic region sequence with an expressed region sequence such as a cDNA or EST will narrow the results essentially to transcribed gene region sequence because such transcribed regions constitute the overlapping set of sequences between the searched pair of seed and target region sequences. The same result also would be obtained when searching a gene with a cDNA or EST, for example, again because the transcribed region of a gene is the common structural region or sequence element between the searched pair of sequences. Similarly, if mRNA region sequence of a gene is to be extended, a seed sequence can be, for example, a cDNA and the target region sequence can be EST sequences. The opposite combination also will achieve a similar result. Finally, as a further example, if coding region sequence is to be the sequence extending information to be produced, then either the seed or the target sequences should be refined to include only coding or exon region sequence.

Therefore, the specificity of the sequence extending information to be obtained will correlate with the genetic structural regions or sequence elements present in the more refined seed or target sequence category of the searched pair. Effectiveness of the methods of the invention can be enhanced, for example, when both the seed and target sequence sources are within similar or the same nucleic acid category. Given the teachings and guidance provided herein, those skilled in the art will know which combinations and permutations of seed and target sequence category pairs can be employed in the methods of the invention to generate any particular category of nucleic acid sequence extending information.

The automated methods of the invention employ a process of simultaneously searching a plurality of nucleic acid target sequences with a plurality of nucleic acid seed sequences. A plurality can include, for example, a wide range of different sized populations. The automated methods of the invention multiplexes populations of nucleic acid seed sequences, nucleic acid target sequences or both to achieve greater computational efficiency in search, alignment and clustering processes and therefore greater sensitivity in output results. Population sizes for either seed or target sequences generally will be in the range of thousands to hundreds of thousands of nucleic acid sequences as such sizes will enable a user to keep up with the newly discovered EST sequences being deposited at a rate of 6,000-8,000 ESTs per day. Similarly, a large scale genomic sequencing facility can have under investigation in any single day hundreds of genomic regions being sequenced, which can correspond to many thousands of genomic region fragments or genes thereof. The automated methods of the invention allows the simultaneous identification of sequence extending information from either or both of these sizes of population on a daily basis or on a larger incremental basis.

Therefore, the automated methods of the invention can efficiently search, identify and select nucleic acid sequence extending information for both small and very large sized populations alike. For example, a plurality can include a group as small as two nucleic acid seed or target sequences as well as groups of hundreds, thousands, ten-thousands, one hundred thousand, hundreds of thousands, one million or greater than a million or more different species of nucleic acid sequences within either or both seed or target sequence populations. Accordingly, pluralities of seed and target sequences can include population sizes of 2, 3, 4, 5, 6, 8, 10, 12, 15, 20, 25, 50 or 100 or more nucleic acid sequences as well as larger population sizes consisting of, for example, a plurality containing 100, 200, 300, 500, 1000, 2000 or 5000 or more different seed or target sequences as well as 6000, 7000, 8000, 10,000, 12,000, 15,000, 20,000, 25,000, 50,000, 100,000, 200,000, 500,000, 1,000,000, 2,000,000, 4,000,000, 5,000,000, 10,000,000 or more different sequences. Pluralities of seed and target sequences include all integer values in between the above-referenced pluralities. It will be apparent to those skilled in the art that the methods of the invention can be applied to an essentially unlimited number of nucleic acid seed or target sequences given the teachings and guidance provided herein. Therefore, the size content of a plurality of seed or target sequences that can be employed in the automated methods of the invention will only be limited by the available computational power.

In selecting a plurality of seed or target sequences for practicing the methods of the invention, it should be understood that one plurality is searched with a query containing the other plurality. As described further below, the searched group is generally referred to herein as the target sequences and the query group is generally referred to herein as the seed sequences. Regardless of the label attached to a particular plurality, it should be understood that one group is searched with a query of the other group. When searching a large number of sequences, greater efficiency can be achieved when the source of the larger of the two pluralities is a database and it is searched with a query of containing the smaller plurality. For example, a database of target sequences containing between about 4-10 million sequences can be searched with a query of about 1,000-2,000 seed sequences in a period of between about 10-16 hours. Although various other formats can be implemented in the methods of the invention, when searching a target sequence database with a smaller plurality of query sequences, the size of the target plurality is unlimited. Results can be obtained in periods of between about 8-18 hours using queries containing between about 500-2,500 seed sequences. Larger size queries of seed sequence pluralities also can be used, including for example, between about 3,000-5,000, generally between about 6,000-9,000 as well as about 10,000 or more seed sequences, although there can be some diminution in computational speed. Therefore, the user can modulate the speed and efficiency of the computation process by adjusting the size of seed sequence pluralities depending on the need and desired outcome.

A plurality of nucleic acid seed or target sequences can be multiplexed to increase efficiency of computer resource use, search algorithms, and computational time. Multiplex, or multiplex analysis, refers to a system that can transmit or analyze several messages or signals simultaneously on the same electronic or digital circuit. The input signal is referred to as a multiplex signal. For example, a multiplexed seed sequence signal can be a data set representing a plurality of seed sequences that can be transmitted together or represented as a single input. Similarly, a multiplexed target sequence signal can be, for example, a data set representing a plurality of target sequences that also can be transmitted together or represented as a single input. Multiplexing therefore reduces the number of sequences in a plurality into a smaller number of data units or element which contain substantially the same information for analysis. Accordingly, a greater number of total sequences can be analyzed in a given time period due to the multiplex reduction in data elements, but not information content. Multiplexing therefore allows the simultaneous searching and computational analysis of pluralities and of groups of pluralities of seed sequences against pluralities and groups of pluralities of target sequences to efficiently obtain nucleic acid sequence extending information for input seed sequences.

One method of multiplexing pluralities of seed and target sequences for use in the methods of the invention is concatenation of such sequences into a single string consisting of multiple different seed or target sequences. The concatenated query sequence is used to search a database as if it were a single nucleic acid sequence. Efficiency is achieved because such concatenated query sequences avoid requiring execution of a new search for each individual sequence contained in the concatenated sequence. One program well known to those skilled in the art for concatenating nucleic acid sequences into multiplex signals for database searches is MPBLAST, Korf and Gish, Bioinformatics, 16:1052-53 (2000), which is incorporated herein by reference. The program is available at the URL:blast.wustl.edu. Other methods for concatenating pluralities of seed or target sequences into multiplex queries include, for example, DeCypher (Timelogic, Inc., Oakland, Calif.), which is available at the URL:timelogic.com.

Briefly, MPBLAST produces multiplex signals by concatenating numerous sequences into a few long sequences in a preprocessor step. The multiplex sequences can then be used as queries in searches such as those involved in local alignment, pairwise alignment, multiple alignment, mapping, clustering and annotating ESTs and genomic DNA fragments. As described previously, such searches can employ, for example, programs such as BLAST, FASTA, d2_cluster, Phrap, and CLUSTAL, as well as any of the specific programs within the family of basic local alignment search tools collectively known as BLAST. For example, BLAST is a heuristic that optimizes a specific similarity measure and can be found described in, for example, Altschul et al. J. Mol. Biol. 215:403-10 (1990), which is incorporated herein by reference. The family of BLAST programs now includes numerous modifications and refinements thereof which are well known to those skilled in the art. Such modifications and refinements include, for example, BLASTN, WU-BLAST, Gapped BLAST, PSI-BLAST and Tera-BLAST. The BLAST family of programs is available at the URL:ncbi.nlm.nih.gov and at URL:blast.wustl.edu. Multiplex queries are particular advantages to increase throughput when used in combination with batch searches such as those performed with BLASTN.

Following a search with a concatenated query, a postprocessor step can be used to parse and deconvolute the results of, for example, an alignment search. Parsing and deconvolution coverts the multiplex query coordinates back to their component sequence origins. To prevent gapped alignments against a multiplex query from crossing individual sequence boundaries a spacer or barrier can be inserted between individual sequences during the concatenation step. A spacer can include, for example, characters such as “N” or “hyphen” that produce negative scores in alignment programs. A spacer also can include a character specifically defined to preclude alignment over it during an alignment process. The spacer should be of sufficient length to terminate gapped alignments before they cross into adjacent sequences in the concatenated string. Such lengths can include, for example, from about 1 to 100 characters, generally from about 1 to 10 characters, and more generally from about 1 to 3 characters. Those skilled in the art will know or can determine the size of the spacer to use between concatenated sequences to ensure termination of a gapped alignment given the search tool used and specific alignment parameters.

The size of a multiplex query can vary from small to a vary large plurality of input sequences. For example, a multiplex concatenated query can range, for example, from 10 bases to millions of bases. The optimal size of a concatenated query can depend, for example, on the available memory on the queried machine, the size of the target database being searched, and the search algorithm employed. The number of distinct sequences assembled in a multiplex query also can influence efficiency, but generally, this factor is inherent in the size, or total number of bases, making up a single concatenated query. For the program BLAST, for example, and using the range of parameters described herein, a multiplex query of between about 50,000-1,000,000 bases, generally about 75,000-750,000 bases, and more generally about 100,000-500,000 bases total can be employed for consistent and efficient results. A multiplex query in the range of about 100,000-500,000 bases corresponds to about 100-1,000 EST sequences or about 1-3 BACs (bacterial artificial chromosomes). Computational power and size generally doubles about every two years. Therefore, multiplex queries can similarly increase in size without loss in efficiency as such computational advancements are made.

Generating a concatenated multiplex query, for example, can be accomplished by assembling the sequences into a single character string as described. The assembly can be performed, for example, sequentially, in parallel or in batch. One specific example is to group the sequences within the query source together in batch sizes that approximate a preselected total length of the multiplex query. The process can additionally include a minimal size cutoff. A pseudocode for such a selection procedure can be:

Initialize current concatenated query set to empty
For each sequence in large query set

Add query to current concatenated query set
If size of query set is greater than chosen size,
then

queue current set for processing
re-initialize current set to empty

endif

end loop

To identify a plurality of nucleic acid sequence extending information for a plurality of nucleic acid seed sequences, one or more queries of a seed sequence plurality can be constructed and used to search against a plurality of nucleic acid target sequences. A query is a user's or agent's request for information, generally as a request to a database or search engine. In the methods of the invention, the request is for a search of a target sequence population and to identify sequences that exhibit significant or substantial alignment to the input query seed sequence data. A specific example of a query that can be used in the methods of the invention can be in formats that include, for example, FASTA, Genbank, EMBL, and plain text sequence, as well as other formats well known to those skilled in the art. As described above, the search queries can be multiplex queries to increase speed, efficiency and use of computational resources. However, the automated methods of the invention can similarly employ non-multiplexed queries to achieve similar results, albeit with less efficiency and greater use of computational resources.

A specific example of a data file that can be employed in the search or other queries of the invention can be, for example, in a FASTA format file (URL:ncbi.nlm.nih.gov/BLAST/fasta.html). The algorithm for FASTA can be found described in, for example, Lipman and Pearson, Science, 227:1435-1441 (1985), which is incorporated herein by reference.

Briefly, a sequence in FASTA format begins with a single-line description, followed by lines of sequence data. The description line is distinguished from the sequence data by a greater-than (“>”) symbol in the first column. It is recommended that all lines of text be shorter than 80 characters in length. An example sequence in FASTA format is:

>gi|532319|pir|TVFV2E|TVFV2E sequence name
ACGGTTCCAAGGCATGCTTCCARYMSTGATCCAAACGCGRYAGGTCAACC

GGHBVGG

AAGGTTCCACGRRCCAATHDGCATTTTTCGCGGGCCGAATCGGCCTATAC

CGGTATA

Sequences are expected to be represented in the standard IUB/IUPAC amino acid and nucleic acid codes, with these exceptions: lower-case letters are accepted and are mapped into upper-case; a single hyphen or dash can be used to represent a gap of indeterminate length; and in amino acid sequences, U and * are acceptable letters (see below). Before submitting a request, any numerical digits in the query sequence should either be removed or replaced by appropriate letter codes (e.g., N for unknown nucleic acid residue or X for unknown amino acid residue). The nucleic acid codes supported are:

| | |
|---|---|
| A −−> adenosine | M −−> A C (amino) |
| C −−> cytidine | S −−> G C (strong) |
| G −−> guanine | W −> A T (weak) |
| T −−> thymidine | B −−> G T C |
| U −−> uridine | D −−> G A T |
| R −−> G A (purine) | H −−> A C T |
| Y −−> T C (pyrimidine) | V −−> G C A |
| K −−> G T (keto) | N −−> A G C T (any) |
| | − −−> gap of indeterminate length |

The above query and file formats, as well as various other formats, are well known to those skilled in the art and can be equally employed in the automated methods of the invention. Given the teachings and guidance provided herein, those skilled in the art will know how to substitute one query or file format for a comparable version. Various choices and combinations thereof will be based on, for example, user preference, computer architecture and computational resources available to the user.

The nucleic acid seed sequences or nucleic acid target sequences can be obtained from any of a variety of sources well known to those skilled in the art. Such sources include for example, user derived, public or private databases, subscription sources and on-line public or private sources. For example, databases for producing a query of seed sequences, or for searching a query of seed sequences against can include, for example, dbEST-human, UniGene-human, gb-new-EST, Genbank, Gb_pat, Gb_htgs, Refseq, Derwent Geneseq and Raw Reeds Databases. Additionally, the source database of the initial input population of seed sequences also can be searched as well. Access or subscription to these repositories can be found, for example, at the following URL addresses: dbEST-human, gb-new-EST, Genbank, Gb_pat, and Gb_htgs at URL:ftp.ncbi.nih.gov/genbank/; Unigene-human at URL:ftp.ncbi.nih.gov/repository/UniGene/; Refseq at URL:ftp.ncbi.nih.gov/refseq/; Derwent Geneseq at URL:www.derwent.com/geneseq/ and Raw Reads Databases at URL:trace.ensembl.org/. The nucleic acid seed or target sequences additionally can be generated by a user source and used directly or stored, for example, in a local database. Various other sources well known to those skilled in the art for obtaining seed or target sequence data also exist and can be similarly be used in the automated methods of the invention.

Multiplex seed sequence queries can be searched, for example, against one or more target sequence databases, either separately or simultaneously. Similarly, seed sequence queries also can be searched separately or simultaneously against one or more seed sequence databases. The number and content size of the target sequence databases that are searched can vary from, for example, a single small database to multiple very large databases. The larger the size of the database content that can be searched, the greater amount of sequence extending information that will be obtained for some or all of the input seed sequences. Similarly, the greater the number of target sequence databases that can be searched, for example, in a given period of time also will identify more sequence extending information for the input nucleic acid seed sequences. Searching seed sequences together with target sequences can result in the further effect of increasing the probability of obtaining sequence extending information as well because it can result in the identification of additional, related seed sequence information. Moreover, searching the input plurality of seed sequences can also serve to identify seed sequences that form part of the same cDNA, gene or genomic region sequence. Given the teachings and guidance provided herein, those skilled in the art will understand that various other combinations and permutations of searches additional to those described above can similarly be conducted simultaneously, in parallel or in series depending on the result to be obtained and available computational resources.

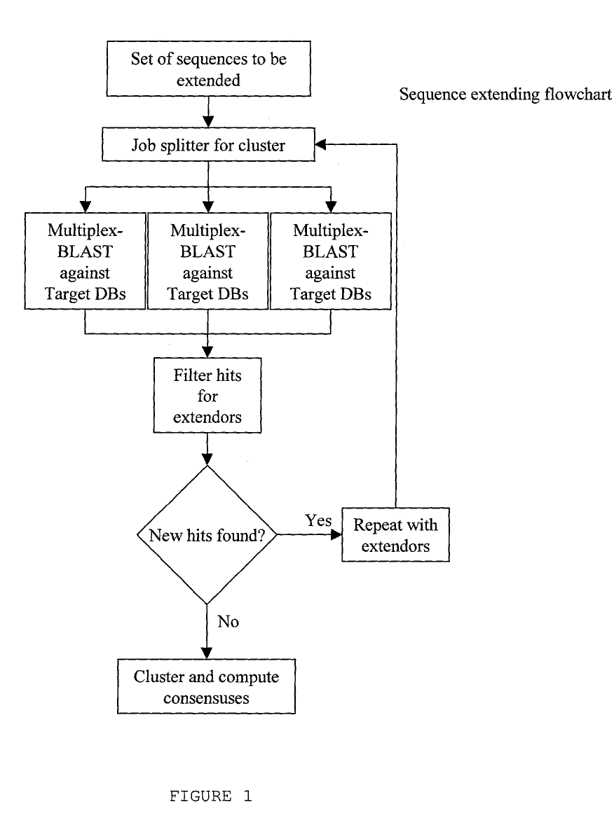

Similarly, searches also can be distributed across computational resources to even the load among available computers or computing clusters. Various methods for load sharing well known to those skilled in the art can be employed. Two such methods include for example, a system based on LSF from Platform Technology that runs WU-BLAST, or a load-balancing system by Timelogic's DeCypher system. Briefly, for a given set of computational resources, one distribution strategy can be, for example, to split searches into sizes roughly proportional to the power of the available resources. Using, for example, LSF, it si sufficient to split the searches up approximately equally and then let the load balancing system send more jobs to the more powerful computers. A flow chart for sequence extension analysis which includes load sharing is shown in FIG. 1.

Identifying a plurality of target sequences that exhibit significant or substantial alignment to the plurality of input query sequence data can be performed by a variety of methods will known in the art. Such method include the search and alignments programs described previously as well as other well known to those skilled in the art. The choice of parameters to set in any particular program used will depend on the level of accuracy desired and search approach chosen for the identification of sequence extending information.

For example, less stringent search parameters can result in the acquisition of more aligned sequences. However, such sequences can be either related or the same as its cognate seed sequence within the query. Additionally, using less stringent parameters can incur greater alignment error, leading to artifacts. Nevertheless, if a user desires to obtain sequence extending information of related sequence, then one option is to employ looser parameters, albeit at the expense of more error. Alternatively, a user can decrease error as well as increase the likelihood of obtaining substantial alignment of seed sequences and target sequences by increasing the stringency of the employed search parameters. Additionally, increasing the stringency of the search parameters can also serve to increase the likelihood of acquiring significant sequence extending information. Further, increasing the stringency of search parameters also will increase the speed of the computation because there will be fewer significant alignments to analyze.

Exemplary search parameters that can be employed for high stringency searches can include, for example, the following values: match=+5, gap=−11, gap extend=−11, and mismatch=−50, S=450 and S2=450, where positive values indicate favorable weighted scores for alignment positions and negative values provide a weighted penalty to the alignment score for gaps, gap extensions and mismatches. S and S2 indicate the score cutoff and the score cutoff for combining two strings, respectively. Setting S and S2 to the same value specifies that the alignment is to be performed in a single path. Such parameters translate into requiring an alignment of about 90 base pairs (bp) without combining smaller subsequences, requires two or more matching positions to compensate for a gap, and further requires about 4-5 matching positions to compensate for a single mismatch. Additional ranges of the above parameters which are sufficient to achieve substantial alignment of seed and target sequences can include, for example, about: match +1, gap −2, gap extend −2, mismatch −10 S 90, and S2 90; generally match +10, gap −22, gap extend −222, mismatch −100 S 900, and S2 900; and more generally match +1, gap −1, gap extend −1, mismatch −2 S 90, and S2 90. Additionally, parameters S and S2 can be varied, without altering the other parameters, to modulate the total number of matching base pairs. For example, S and S2 can be set at about 250 to obtain between an about 50-100 bp alignment. Setting S and S2 at about 5000 can achieve an about 1000 base or pair base alignment.

Alternatively, ranges of the above parameters for moderate stringency can include, for example, about: match +10, gap −22, gap extend −222, mismatch −40 S 900, and S2 900. Ranges of the above parameters for preforming gene discovery analysis, as described further below, can include, for example, about: match +1, gap −1, gap extend −1, mismatch −2 S 64, and S2 13, generally match +1, gap −1, gap extend −1, mismatch −2, S 90 and S2 90, and more generally, match +5, gap −11, gap extend −11, mismatch −22, S 200, and S2 200. Moveover, when performing the methods to extend BAC sequences the S and S2 parameters can be varied to a range of about 1000-6000, which would require an alignment of about 200-1200 bases.

The scoring matrix that can be used in the search and alignment process can be, for example, BLOSUM62, or other equivalent forms well known to those skilled in the art. Given the teachings and guidance set forth above, those skilled in the art will know, or can determine parameters for other search and alignment programs given the teaching and guidance provided herein.

To increase the likelihood of acquiring significant sequence extending information, parameters specifying a minimum length of overlap between aligned seed and target sequences can be employed. Moreover, other parameters also can be employed which specify, for example, a minimum length of single-stranded overhang. Single-strand overhang sequence constitutes one form of sequence extending information identified by the methods of the invention. For example, to obtain a substantial alignment between seed and target sequences, the search parameters can specify a minimum number of aligned bases of between about 12-25 bases. More stringent alignments can include between about 26-50 bases or about 75 bases. Very stringent alignments can require about 90 bases or more to be substantially aligned between a seed and target sequence before the target sequence is selected for determination of its sequence extending information.

An additional search parameter that can be employed to increase stringency of alignment results can be, for example, to select only those substantially aligned target sequences that match to within a minimum number of residues from target sequence's internal terminus. This parameter compensates for low quality terminal sequence information inherent in many genomic sequence data. Therefore, the greater the extent of match in the overlapping region of seed and target sequence, the greater the likelihood of incurring less error. A minimal number of sequences can be, for example, about 50 bases, generally about 25 bases, and more generally about 10 bases or less.

To increase the percentage of identified substantially aligned target sequences that have productive amounts of nucleic acid sequence extending information, a further parameter can be employed which preferentially selects those substantially aligned sequences that result in a minimum amount of extending sequence. For example, the more stringent the search parameter, the greater the length of sequence extending information that will be obtained. However, increasing length of such extending sequence, or single-stranded overhang sequence, can concomitantly decrease the number of target sequences with sequence extending information fitting the search criteria. A balance can be obtained that increases overall efficiency by selecting and extending from a greater number of extending target sequences having significant, but shorter, lengths of sequence extending information. For example, a minimum single-stranded overhang length parameter can be employed that specifies a minimum length about 20 bases of sequence extending information, generally about 40 bases, and more generally about 50-100 bases or more.

Following searching a plurality of target sequences with one or more queries of a plurality of seed sequences and identifying at least two or more target sequences that substantially align with at least two or more seed sequences as described above, the automated methods of the invention can select, for example, some or all of those target sequence that contain sequence extending information. The logic for of the selection can be, for example, to select any sequence that contains sequence information extending past either or both termini of its cognate seed sequence. Alternatively, as described above, only those target sequences that contain a specified amount of sequence information, or length of overhang, can be selected. The sequence content within the extending portion or overhang region constitutes nucleic acid sequence extending information of the invention.

The plurality of target sequences containing sequence extending information can be selected and output for user review or analysis. Alternatively, further sequence extending information can be acquired by collecting the selected plurality of substantially aligned target sequences containing sequence extending information and then employing those target sequences as a new plurality of nucleic acid seed sequences. In this manner, a user can repetitively walk into adjacent or contiguous regions of seed sequence nucleic acid segments to obtain additional quantities of sequence extending information. Additionally, the new search query composed of the selected aligned target sequences can be performed, for example, either alone or in combination with the original plurality of seed sequences. The process can be repeated with each selected plurality of substantially aligned target sequences, or combinations thereof, as a new input pluralities of seed sequences until the no further sequence extending information is identified. Exhausting the identification process can indicate that much, if not all, of the available database sequence information has been gathered and identified to its cognate seed sequence.

Therefore, the invention provides an automated method of simultaneously identifying nucleic acid sequence information extending a plurality of nucleic acid seed sequences wherein the steps of searching, identifying and selecting a plurality of substantially aligned target sequences containing nucleic acid sequence extending information is repeated two or more times with the selected target sequences being used as a nascent plurality of seed sequences.

The invention also provides an automated method for simultaneously identifying unidirectional or bidirectional sequence extending information for a plurality of seed sequences. The method consists of selecting those substantially aligned target sequences containing either unidirectional or bidirectional nucleic acid sequence extending information.

Unidirectional extension can be desirable when, for example, an orientation is known or a terminal portion of the seed sequence is complete or otherwise irrelevant. Bidirectional extension can be desirable when, for example, a seed sequence has an unknown orientation, is believed to be incomplete or when sequence extending information is to be maximized. To obtain unidirectional sequence extending information a search parameter can be employed to select only those substantially aligned target sequences that contain sequence extending information at a single 5′ or 3′ terminus. The logic for unidirectional extension of a plurality of seed sequences can be, for example: select target sequences aligning with X bases of each seed sequence Nᵢ within plurality Nᵢⱼ. An alternative logic for unidirectional extension of a plurality of seed sequences can be, for example: select target sequences aligning with X bases AND NOT with Y bases of each seed sequence Nᵢ within plurality Nᵢⱼ. An example of a pseudocode for such a procedure can be:

Given a sequence (S) to be extended, and a set of
directions D (S) that sequence needs extensions for (“5′”,
“3” or “5′, 3′”)
For each hit H of S which matches the alignment
parameters specified (i.e. has at least a certain score
given the match, mismatch, and gap penalties)
For each direction in set D (S)

If H extends S in the given direction, add it to
set of extending sequences for S E (S).

end loop on directions
end loop on hits
return set E (S)

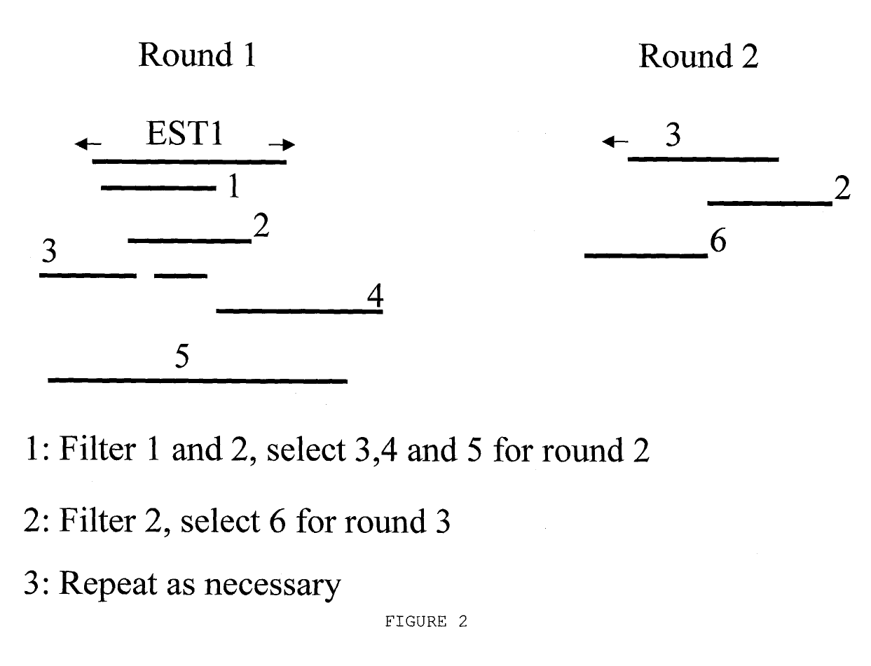

To obtain bidirectional sequence extending information all that is needed is to select the plurality of target sequences that substantially align with both 5′ and 3′ termini of the seed sequence plurality. A specific example of bidirectional extension is shown in FIG. 2 where those target sequences obtained in the initial search containing sequence extending information in both directions are selected as nascent seed sequences for a subsequent search round. As will be described further below, FIG. 2 also shows a pruning process where superfluous internal, redundant sequence information is eliminated from subsequent search rounds.

The invention additionally provides an automated method of simultaneously identifying nucleic acid sequence information extending a plurality of nucleic acid seed sequences wherein superfluous nucleic acid seed sequence or target sequence is pruned. Pruning superfluous nucleic acid sequences provides particular advantages of reducing computational load and increasing the efficiency of computational resources. Accordingly, the speed, accuracy and sensitivity of the results also are enhanced.

As described previously, superfluous nucleic acid sequence information consists of sequence information that is dispensable or nonessential for executing one or more steps in the methods of the invention. Such dispensable or nonessential information can include, for example, information that is unnecessary, unwanted or redundant. By pruning or eliminating such information, subsequent rounds of querying pluralities of multiplex seed sequences against pluralities of target sequences will contain mostly essential or relevant information in each nascent search query.

Pruning can be accomplished by a variety of processes well known to those skilled the art. One process of particular use in the automated methods of the invention includes, for example, filtering, removing, deselecting or masking substantially aligned target sequences that have been previously selected for containing sequence extending information. Target sequences that can be pruned include, for example, those that are redundant with other selected target sequences or those that contain overlapping sequence information internal to one or both termini of its cognate seed sequence.

Pruning such redundant, internal or partially internal substantially aligned target sequences can be performed, for example, during the first round of searching and selection or during any and all subsequent rounds. Additionally, pruning also can be performed on pluralities of seed sequence. For example, where a seed sequence is sufficiently long, it can be beneficial to prune the internal portion sequence to enhance extension results. Similarly, where rounds of extension have generated substantial sequence extending information outside the termini of a seed sequence, that seed sequence can be subsequently removed from further analysis.

A substantially aligned target sequence that contains overlapping sequence internal to only one terminus of its cognate seed sequence will, for example, terminate at the same position as its seed sequence or contain sequence extending information. In the former case, the target sequence will contain only redundant information and therefore beneficial to prune. In the latter case, it can be desirable to prune if unidirectional extension of the opposite terminus is objective to be obtained.

Other categories of selected substantially aligned target sequences that can be labeled as superfluous and pruned includes, for example, substantially abundant aligned target sequences and substantially overabundant target sequences. The former category can include, for example, aligned target sequences containing about 200, generally about 400, more generally about 500 or more alignments with a cognate seed sequence. The latter category can include, for example, aligned target sequences of about 8,000, generally about 10,000, and more generally about 12,000 or more alignments with a cognate seed sequence or members within a single cluster grouping corresponding to one or a few seed sequences.

The above described substantially abundant and overabundant pruning categories represent general classes of repetitive nucleic acid sequences. The automated methods of the invention cure such inefficiencies by setting a cutoff limit at a level that is considered to represent superfluous information. One cutoff limit is relative to the number of alignments with a seed sequence and the other cutoff limit is relative to the total number of target sequences that can be grouped as an extension product of a seed sequence. The former referring to a substantially abundant target sequence and the latter referring to overabundant members within a seed sequence cluster.

Other categories of substantially abundant and overabundant sequences well known to those skilled in the art similarly can be marked for pruning as superfluous nucleic acid sequence information and removed from initial or subsequent analysis. Such other superfluous sequences can result from, for example, similarity in sequence due to structure or function, such as poly A tails, centromeric or teliomeric region sequence. Additionally, sequence similarity that can be desirable to prune can result when performing the sequence extension methods of the invention with pluralities of seed or target sequences containing unwanted paralog and ortholog sequences. Given the teachings and guidance provided herein, those skilled in the art will known, or can determine an appropriate pruning step to filter, remove, mask or otherwise eliminate some or all of essentially any undesirable sequence or fragment thereof.

The logic for pruning redundant target and substantially abundant or overabundent sequences is similar to that for selecting unidirectional target sequence extending information. For example: select target sequences aligning with X bases of each seed sequence Nᵢ within plurality Nᵢⱼ, or alternatively, select target sequences aligning with X bases AND NOT with Y bases of each seed sequence Nᵢ within plurality Nᵢⱼ. An example of a pseudocode for such pruning procedures can be:

Initialize set E of sequences to perform extensions on
and needed directions to the set of (Seed, “5′, 3′”) fo
reach of the initial seeds. E = {(s,d), d = “5′, 3′” for
each s in Seeds}
Begin iteration.
Perform multiplex search with query set consisting of all
the sequences s in set E
For each sequence s, prune out all the hits which do not
extend s in one of the directions in its paired direction
d. (Direction-based pruning)
If there are fewer directional-extending hits left than
the per-sequence pruning cutoff (e.g. 500), add them to
the set E′ of hits to be extended in the next iteration.
Each hit's direction will be 5′ if in the 3′ direction,
or “5′, 3′” if it extended its query in both directions.
If the entire set of hits for a single initial seed it
greater than the cluster-size cutoff (e.g. 12000), remove
any of its hits from E′
Let the set E′ become set E for the next round of the
iteration
If E′ is not empty, repeat the iteration

Other methods of filtering, removing, deselecting or masking are well known in the art.

The selected, or selected and pruned, plurality of substantially aligned target sequences can additionally be clustered, for example, during or at anytime following the initial round of searching and selection. Clustering refers to grouping of sequences. Computationally, clustering entails the process of partitioning nucleic acid sequence data into index classes, or clusters, where each class represents the same nucleic acid. Generally, clustering is preformed with reference to genes and each index class represents a different gene such that cDNAs, ESTs, ORFs and the like are partitioned into the same index class if they contain sequence representing the same gene.

Clustering pruned target sequences allows for the selection of a single target sequence species from the group of substantially aligned target sequences corresponding to a single cognate seed sequence. The selected target sequence species can then be used, for example, as a nascent seed sequence in subsequent rounds of extension. The sequence species to select will generally be a representative sequence of the cluster. Such representative sequence species can be, for example, a consensus sequences, a sequence representing the 5′ end of the cluster or a sequence representing the 3′ end of the cluster. The choice of representative sequence specie to select will depend on the need and objective of the user. Accordingly, sequences other than a consensus or terminal region sequence also can be selected. Therefore, By choosing a single representative sequence from among a group, clustering enhances efficiency during subsequent rounds because in reduces data load within the nascent multiplex query by removing unnecessary or redundant sequence information. The process of clustering further allows efficient selection of the most terminal selected plurality of substantially aligned target sequences relative to its cognate seed sequence because of the inherent logic in a cluster grouping. In this regard, it is computationally simpler to select the most terminal target sequences within a contiguous set of overlapping sequence strings without having to string searches or other comparable analysis.

There are various automated methods well known to those skilled in the art which can perform clustering processes. Such programs include, for example, d2_cluster, THC_BUILD, and UniGene. A description of d2_cluster can be found described in, for example, Burke et al., Genome Res., 9:1135-42 (1999), and is available through Double Twist, Inc. (formerly known as Pangea Systems, Oakland, Calif.) at the URL:pangeasystems.com. THC_BUILD can be found described in, for example, Adams et al., Nature (Suppl.) 377:3-17 (1995); Sutton et al., Genome Sci. Technol. 1:9-18 (1995), and in White and Kerlavage, Meth. Enzymol., 206:27-41 (1996). THC_BUILD is available through The Institute for Genomic Research (TIGR) (Rockville, Md.) and at the URL:tigr.org/hgi/hgi_info.html. Finally, UniGene can be found described in, for example, Boguski and Schuler, Nat. Genet., 10:369-71 (1995) and in Schuler et al., Science, 274:540-46 (1996), which is available through the National Institutes of Health at the URL:ncbi.nlm.nih.gov/UniGene/TXT/build.html. All of the above cited references are incorporated herein by references.

Although algorithms can differ between clustering programs, the logic for each is to form index classes based on sequence similarity. With reference to d2_cluster, for example, this program employs an agglomerative algorithm that partitions sequence data into index classes according to minimal linkage rules. Briefly, every sequence begins in its own cluster and the final clustering is constructed through a series of mergers. Minimal linkage rules, which also is referred to in the art as single linkage or transitive closure, refers to the property that any two sequences with a given level of similarity will be in the same cluster. For example, two sequences will be in the same cluster even if they share no sequence similarity if there exists a third sequence that exhibits sufficient sequence similarity to both the first and second sequence. Therefore, the only criterion for clustering with d2_cluster is sequence overlap.

Clusters resulting from the simultaneous searching of a plurality of target sequences with a plurality of seed sequences can be assembled into representations corresponding to a single contiguous nucleic acid sequence representing the merger of the initial seed sequence and its resulting extension products. The sequence information additional to the initial seed sequence represents nucleic acid sequence extending information of the invention.

Additionally, one or more assembly products, their clusters, or subcomponents thereof, can be assembled against genomic region sequence to map its genomic location. Depending on the source of the plurality of seed sequences, the genomic region sequence can corresponding to the initial seed sequences or be mapped de novo. One program well known to those skilled in the art for assembling nucleic acid sequences with genomic sequences is sim4 (Double Twist, Inc. (formerly known as Pangea Systems), Oakland, Calif.) and is available at the URL:pangeasystems.com. The program Double Twist CAT, which includes sim4, can be found described in, for example, Florea et al., Genome Res., 8:967-74 (1998), and Burke et al., supra, which are incorporated herein by reference. Other assembly programs well known in the art can similarly be used for assembling selected substantially aligned target sequences containing sequence extending information, their pruned products or their clustered groups.

Therefore, the invention provides an automated method of simultaneously identifying nucleic acid sequence information extending a plurality of nucleic acid seed sequences wherein a plurality of consensus nucleic acid target sequences are generated by clustering selected target sequences containing sequence extending information. The plurality of consensus sequences can be merged with their cognate seed sequences to produce one or more extended nucleic acid seed sequences.

Also provided is an automated method of simultaneous identifying a plurality of gene sequences within a plurality of genomic region sequences. The method consists of: (a) pruning nucleic acid sequence elements from a plurality of genomic region sequences to produce a plurality of genomic seed sequences; (b) searching a plurality of target gene sequences with a multiplex query comprising a plurality of genomic seed sequences; (c) identifying a plurality of target gene sequences substantially aligning with a plurality of genomic seed sequences, and (d) locating regions of substantial alignment of the identified plurality of target gene sequences within the plurality of genomic region sequences, the regions of substantial alignment identifying a plurality of gene sequences.

The method further provides for obtaining gene specific nucleic acid sequence extending information within adjacent genomic region sequences for the identified plurality of gene sequences. The method consists of: (a) searching a plurality of nucleic acid target sequences with a multiplex query comprising a plurality of gene seed sequences; (b) identifying a plurality of target sequences substantially aligning with a plurality of gene seed sequences; (c) selecting a plurality of substantially aligned target sequences containing nucleic acid sequence extending information for a plurality of gene seed sequences, and (d) repeating steps (a) through (c) using the selected plurality of substantially aligned target sequences as a plurality of gene seed sequences.

As described previously, the automated methods of the invention also can be used for gene discovery and analysis. For example, genomic region segments can be interrogated with a variety of different categories of nucleic acid fragments to discover, or newly identify, previously unrecognized gene regions within a genomic region sequence. Gene structure can be parsed, for example, by interrogating its nucleic acid sequence with exons, introns, untranslated region sequences, promoter or regulatory region sequences, intragenic region sequence and combinations thereof. As described previously, those skilled in the art will know what categories of nucleic acid sequence to use as a seed and target sequence to achieve a particular result. With the availability of a vast amount of sequence information archived in databases, the automated methods of the invention allow the discovery and characterization of essentially an unlimited number of genes, other genetic structural regions as well as domains or fragments thereof, within a gene, genomic region, chromosome or genome.

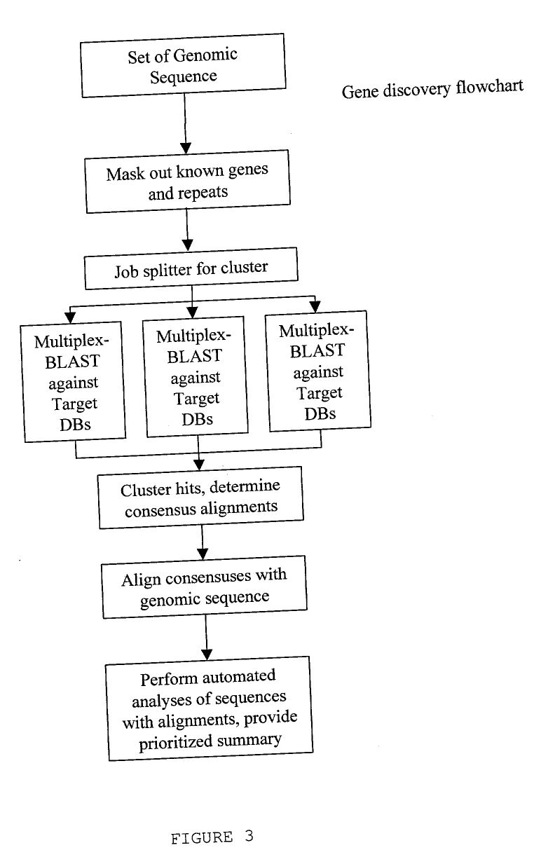

Briefly, with reference to gene discovery and characterization, the automated methods of the invention can be employed to identify existing but as yet unrecognized genes within, for example, a genomic region sequence. Discovery of a new gene within a genomic region sequence can be performed by employing the genomic region sequence directly as a seed sequence. Alternatively, genomic region sequences can be pruned to increase computational efficiency. A flow chart for gene discovery analysis which includes pruning and local sharing is shown in FIG. 3.

Sequences that are superfluous for gene discovery and identification include, for example, those nucleic acid sequences and elements within a seed sequence that are known to correspond to a gene or other structural region. Such elements and regions can be, for example, completely identified gene sequences, partially identified gene sequences, completely or partially identified intragenic regions sequences, completely or partially identified repetitive sequences, as well as fragments thereof. Other sequences known to those skilled in the art which correspond to recognized genes or other structural regions or elements can similarly be pruned to eliminate their inclusion within a seed sequence query of the invention.

Completely identified elements or regions include, for example, those sequence where the 5′ and 3′ structural region boundaries have been identified. Partially identified elements or regions can include, for example, those sequences where only the 5′ or 3′ boundary of the structural region has been identified. Pruning completely identified elements or regions can be accomplished by removing the entire known sequence. On the other hand, when pruning partially identified elements or regions, it can be beneficial to leave some of the known sequence within either or both termini in the event a cryptic boundary has yet to be identified. Other selections for pruning completely or partially identified nucleic acid sequence elements or regions are well known to those skilled in the art and also be employed equally well or in conjunction with those methods described herein.

Superfluous nucleic acid sequence elements can be pruned, for example, prior to assimilating the seed sequences into single or multiplex queries for searching against target sequences. Alternatively, individual or multiplex queries can be generated and then processed to remove superfluous sequences. Initially removing superfluous sequence can be advantageous for reducing computational load and use of computer resources.

Pruning can be accomplished using, for example, the processes described previously with respect to the identification of nucleic acid sequence extending information. Briefly, superfluous nucleic acid sequence elements or regions can be eliminated from the analysis by, for example, filtering, removing, masking and deselection of completely or partially identified elements or regions as well as other superfluous sequences contained in the seed sequences. As described previously, such processes are well known to those skilled in the art and can be accomplished either manually or by various computational methods well known in the art.

Identification of a gene sequence within a genomic region can performed by searching a plurality of target gene sequences. As described previously, the target sequences can be any nucleic acid category. It can be particularly efficient to use target sequences that contain known or recognizable gene region sequence. By inclusion of known or recognizable gene region sequence in the plurality of target sequences, any substantial alignments identified will indicate the presence of a gene. Target gene sequences can include nucleic acid sequence corresponding to, for example, cDNAs, ESTs, exons, introns, untranslated regions, promoter regions, regulatory regions, 5′ flanking regions, or 3′ flanking regions. Therefore, the methods of the invention can employ any of a variety of categories of nucleic acid sequence information, including various combinations and permutations thereof, as target gene sequences for the discovery and characterization of genes within a plurality of genomic seed sequences.

The automated methods for identifying a plurality of gene sequences with a plurality of genomic sequences can be performed similarly to the methods described previous for the identification of nucleic acid sequence extending information. Briefly, the method employs searching a plurality of target gene sequences with, for example, a multiplex query of pruned genomic seed sequences and identifying those target sequences exhibiting substantial alignment with their cognate seed sequences. The search and selection parameters employed will be similar to those described previously with respect to identifying extension products for sequence extending information.

For example, stringent search parameters can substantially increase the likelihood that a target sequence corresponding to, or deemed to correspond to, the same gene represented in the genomic seed sequence will be identified. Similarly, overlap and overhang parameters can be employed to further increase stringency of the search and selection. Alternatively, lower stringent conditions can be employed to obtain more hits, as represented by significant alignment, albeit with an increased likelihood of miscorrespondence between target and seed sequence. Those target sequences identified to substantially align with a genomic seed sequence indicates that the genomic seed sequence contains at least one gene. Alignment of the identified substantially aligned target gene sequence with its cognate genomic seed sequence can then be performed to identify the location of the gene nucleic acid sequence.

In addition to identifying genes within genomic seed sequences, the automated methods of the invention also can be used, for example, to obtain nucleic acid sequence extending information from the new gene sequences once identified. As described previously, sequence extending information can be obtained for essentially any plurality of nucleic acid seed sequences by identifying those substantially aligned target sequences that contain sequence information additional to the plurality of seed sequences. The additional information can extend the sequence at one or both termini of its cognate seed sequence, or alternatively, it can extend it internally, within for example, an intron or alternative splice region. Therefore, a plurality of gene sequences identified from input genomic seed sequences can additionally be used as nascent gene seed sequences in the automated methods of the invention to obtain nucleic acid sequence extending information. The method can be repeated one or more times to acquire additional sequence extending information or iteratively repeated until the identification of sequence extending information has been exhausted.

The choice of target sequence plurality to employ will depend on whether terminal or internal sequence extending information is to be obtained. For example, if unidirectional or bidirectional terminal sequence extending information is to be obtain, then those substantially aligned identified gene sequences located at the one terminus, or both termini, respectively, can be selected and used in further extension rounds as nascent gene seed sequences. Similarly, if internal sequence extending information is to be obtained, then those substantially aligned identified gene sequences located within an internal region of its cognate seed sequence can be selected and used in further extension rounds as nascent gene seed sequences. Specific examples of internal sequence extending information would be sequence information that extends from an exon into an intron, from an intron into an exon, that crosses alternatively spliced junctions, and that identifies internal sequencing artifacts. Other examples of internal sequence extending information are similarly included and which are well known in the art.

The selection of unidirectional, bidirectional or internal sequence extending information can serve to identify specific directional sequence extending information as well as to prune superfluous sequence information. As described previously, by selecting a portion of substantially aligned target gene sequences, those other sequences that substantially align but lack productive information are pruned from subsequent rounds of extension or discovery. Therefore, the process described previously for employing high, moderate or low stringent search parameters, employing overlap and overhang parameters and for pruning superfluous sequence information are equally applicable for the discovery of gene sequences within a plurality of genomic regions and for their subsequent extension.

Also, as described previously in regard to sequence extension analysis, the automated methods for identifying sequence extending information for pluralities of identified gene sequences or nascent gene seed sequences similarly can cluster substantially aligned target sequences containing sequence extending information to obtain a plurality of consensus target sequences. Each consensus sequence within the plurality will correspond to a cognate gene seed sequence. The plurality of consensus target sequences can be, for example, used directly as nascent gene seed sequences or aligned with their cognate gene seed sequences to produce a merged primary sequence containing the sequence extending information. Any or all part of the extended gene seed sequences thus produced can be employed in, for example, subsequent rounds of extension. Additionally, the some or all of the plurality of identified substantially aligned target sequences containing sequence extending information, or their consensus sequences obtained from clustering can be, for example, further aligned with the original plurality of genomic region sequences to map the location of these newly discovered genes within the genomic region sequence.

Once a gene has been identified and mapped to its genomic location, subsequent rounds of either gene discovery or sequence extension can be performed, for example, with uncharacterized adjacent genomic region sequence. As described previously, the process can continue following any or all of the automated methods described above until all desired regions have been characterized or interrogated for new genes or for obtainment of sequence extending information. Additionally, the newly discovered genes can be further characterized by methods well known to those skilled in the art. Such further characterizations can include, for example, the identification of any of various sequence elements and domains well known to those skilled in the art. Sequence elements and domains that can be searched for and annotated on the gene sequence include, for example, promoters, enhancers, intron splicing signals, other processing signals, poly-A signals, poly-A tails, start codons, stop codons, transcription start sites, as well as other expression, regulatory, or binding domain sequence elements.

Therefore, the invention provides an automated method of simultaneously identifying a plurality of gene sequences within a plurality of genomic region sequences and obtaining nucleic acid sequence extending information within adjacent genomic region sequence specific for the plurality of identified gene sequences. The method consists of: locating regions of substantial alignment of an identified plurality of target gene sequences withing a plurality of genomic region sequences, the regions of substantial alignment identifying a plurality of gene sequences, and obtaining gene specific nucleic acid extending information within adjacent genomic region sequences for the identified plurality of gene sequences. Extension can be performed either with or without first clustering the substantially aligned target sequences and with or without mapping of sequence extending information back to genomic regions sequence.

The invention also provides a system for automated discovery of sequence extending information and gene or polypeptide sequences. The automated methods describe previously can be assembled in a suite, for example, of written instructions for use in any of the various computer and computer operating systems available to those skilled in the art. Additionally, the system can contain, for example, all or some of the various functions described herein to allow a user to implement such methods on their particular computer machinery or with their preferred software. The system can be, for example, written code or contained in a computer readable medium and furnished as software. Alternatively, the system of the invention can be embedded into hardware components as operating or peripheral software for such a device. Therefore, an automated extension or discovery system of the invention can include, for example, written code, software, a processor, peripheral, computer, computational cluster or other system programmed to execute some or all of the methods of the invention.

It is understood that modifications which do not substantially affect the activity of the various embodiments of this invention are also included within the definition of the invention provided herein. Accordingly, the following examples are intended to illustrate but not limit the present invention.

### EXAMPLE I

### Multiplex Gene Extension Analysis Using an EST Database

This Example describes the identification of sequence extending information from the simultaneous extension of a population of seed sequences.

An automated extension analysis was performed on a population of EST seed sequences. Briefly, the implementing program uses as input of one or more FASTA-formatted seed files, which can contain the seeds of a clustering process. Each FASTA file represents a single EST or single cluster, and the first entry in the FASTA file is considered to be the seed of that file. Therefore, if different sequences in a cluster are to be used for the extension process, they should be in different FASTA files.

The algorithm for the automated extension process is as follows: For each seed, search it against one or more specified databases. The default databases are dbEST-human and unigene-human searched together with a database consisting of all input EST seed sequences. Search for substantial alignments which overlap significantly by requiring about 90 base matches or more and extend the seed sequence in either or both directions by at least a minimum overhang length. The default minimum overhang length is 40 bases. Track which direction the substantial alignment extends the seed, and therefore which direction a substantial alignment should be extended. Repeat the search, until no new substantial alignments are identified.

Once no further substantial alignments are identified, the automated process will generate FASTA format files for each seed sequence. Each FASTA file consists of all the target sequences which extend it, and will submit any non-singleton clusters to Double Twist CAT for clustering. Results are placed in a specified directory. For convenience, the clustering results are submitted to a load balancing system such as LSF in order of increasing cluster size, which allows viewing an further analysis of the smaller clusters without having to wait for the larger clusters to finish.

The implementing program saves its program state in a work directory so that it is possible to quit and restart the program. When restarted, the implementing program will resume proceedings from about where it left off. To start proceed with a new analysis, the suspended proceedings can be cleared, the work directory can be deleted or the user can specify a different directory.

Sometimes a substantial alignment will extend into a repeat region, or will extend to a region with too many hits for efficient processing continue. The implementing program can prune such substantially abundant alignments and substantially overabundant clusters. Substantially abundant alignments are pruned by identifying single seed sequences having about 500 or more substantially aligned target sequences. Such substantial alignments will not be further extended and are eliminated from further analysis. Substantially overabundant groupings are pruned if after any iteration of alignments a single cluster has more than 12,000 target sequences. Such clusters will be flagged as “too large” and their consensus sequences will be eliminated from further iterations.

The implementing program of the above algorithm, termed “EST extend” was used to obtain sequence extending information for a population of nucleic acid seed sequences that corresponded to fragments of known gene sequences. Briefly, full-length sequence of 28 well-known genes were selected from NCBI refseq (URL:www.ncbi.nlm.nih.gov/LocusLink/refseq.html). These full-length gene sequences were used as both a comparison with the final extension results and to obtain corresponding fragments to use as seed sequences.

To obtain seed sequences for each of the full-length gene sequences, all the ESTs that make up these full-length sequences were first extracted from the NCBI unigene.all database. The shortest EST corresponding to each gene was used as a seed sequence for extension analysis.

To simultaneously identify sequence extending information for each of the 28 seed sequences, iterative rounds of querying the default databases with the population of seed sequences was performed as described above. Search parameters that were employed had the following values: match=+5, gap=−11, gap extend=−11, and mismatch=−50, S=450 and S2=450. Target EST sequences containing overhang sequence information which exhibited substantial alignment to the seed sequences were selected and used in subsequent search rounds as nascent seed sequences. The total time for the extension procedure for the 28 seed sequences was about 10 hours when run on a 3 computer, 12-CPU cluster which was not dedicated to this task. Therefore, there were other programs being run simultaneous on this computational cluster. During the extension analysis, a total of 5 clusters were pruned as being substantially overabundant by using the greater than 12,000 cluster-size cutoff parameter. A total of 4 clusters had some of their alignments pruned as being substantially abundant by using the greater than 500 individual targets extender cutoff, resulting in a total of 6,972 pruned alignments.

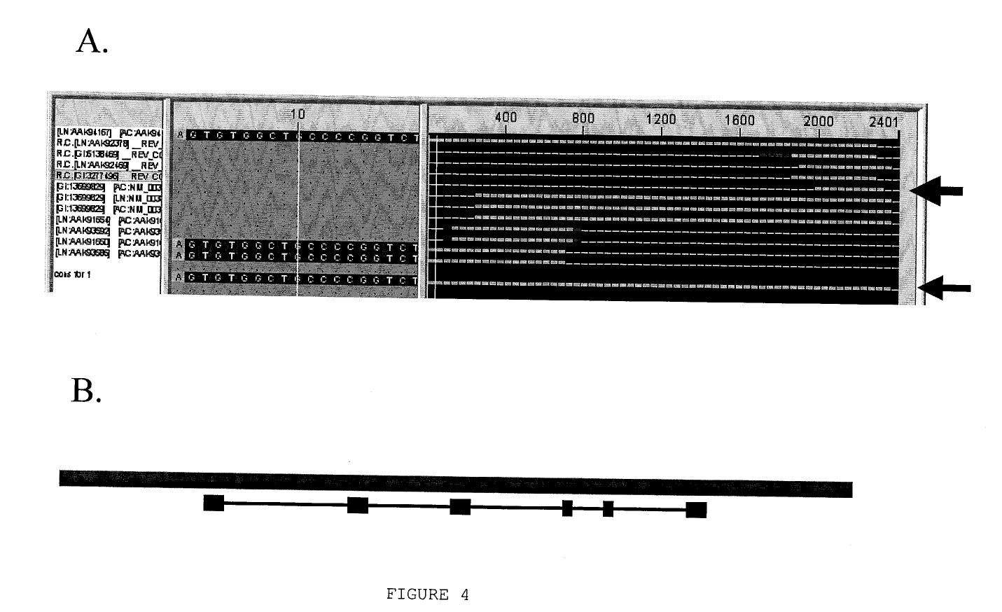

The consensus sequence generated for each seed sequence from the extension analysis was used to compare with the known full length sequences. Out of the 23 seed sequences which were not pruned as being substantially overabundant, 21 generated consensus sequences that were 100% identical to known full-length sequence. The other 2 seed sequences generated consensus sequences that were 99% and 97% identical to the known full-length sequence. FIG. 4A shows a graphic view for the extension results for one of the 23 seeds that was analyzed. The sequence pointed to by the black arrow corresponds to the EST seed sequence and the sequence pointed to by the red arrow (grey arrow in non-color figure) corresponds to the obtained consensus sequence. The sequence extending information obtained from the analysis for this seed was 100% identical to the known full-length sequence. FIG. 4B shows a graphic view of the genomic structure of this gene generated following alignment to its corresponding gene sequence by the EST_extend program. As shown, this particular gene contains six exons.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

What is claimed is:

1. An automated method of simultaneously identifying sequence information extending a plurality of seed sequences, comprising:
(a) searching a plurality of target sequences with a multiplex query comprising a plurality of seed sequences;
(b) identifying a plurality of target sequences substantially aligning with a plurality of seed sequences;
(c) selecting a plurality of substantially aligned target sequences containing sequence extending information for a plurality of seed sequences, and
d) repeating steps (a) through (c) using said selected plurality of substantially aligned target sequences as a plurality of seed sequences.

2. The method of claim 1, further comprising repeating step (d) one or more times.

3. The method of claim 2, further comprising repeating said step (d) until identification of sequence extending information for said plurality of seed sequences is exhausted.

4. The method of claim 1, further comprising selecting substantially aligned target sequences containing unidirectional sequence extending information.

5. The method of claim 1, further comprising selecting substantially aligned target sequences containing bidirectional sequence extending information.

6. The method of claim 1, further comprising identifying nucleic acid target sequences substantially aligning with about 90 base pairs (bp) or more of seed sequence.

7. The method of claim 1, further comprising selecting substantially aligned nucleic acid target sequences having about 40 bases (b) or more of sequence extending information.

8. The method of claim 1, further comprising pruning superfluous sequence information from said plurality of seed sequences or target sequences.

9. The method of claim 8, wherein said pruning is selected from the group of filtering, removing and masking of sequence information.

10. The method of claim 8, wherein said superfluous sequence information further comprises substantially abundant target sequences.

11. The method of claim 10, wherein said substantially abundant target sequences comprise about 500 or more substantial alignments with a seed sequence.

12. The method of claim 8, wherein said superfluous sequence information further comprises substantially overabundant members within a target sequence cluster.

13. The method of claim 12, wherein said target sequence clusters comprise greater than about 12,000 or more members.

14. The method of claim 8, wherein said superfluous sequence information further comprises internal or terminal sequence information.

15. The method of claim 8, wherein said pruning results in bidirectional or unidirectional identification of sequence extending information.

16. The method of claim 1, wherein said plurality of target sequences are selected from the group consisting of expressed sequence tags (ESTs), cDNA, genomic DNA, read nucleic acid sequence, and polypeptide, or fragments thereof.

17. The method of claim 1, wherein said plurality of seed sequences are selected from the group consisting of expressed sequence tags (ESTs), cDNA, genomic DNA, read nucleic acid sequence, and polypeptide, or fragments thereof.

18. The method of claim 1, wherein said multiplex query further comprises a concatenated plurality of seed sequences.

19. The method of claim 18, further comprising deconvoluting said identified plurality of target sequences into component target sequences.

20. The method of claim 1, further comprising the step of clustering the selected plurality of substantially aligned target sequences containing sequence extending information to obtain a plurality of consensus target sequence.

21. The method of claim 20, further comprising aligning one or more of said plurality of consensus target sequences with one or more of said plurality of seed sequences to produce one or more extended seed sequence.

22. An automated method of simultaneous identifying a plurality of gene sequences within a plurality of genomic region sequences, comprising:
(a) pruning nucleic acid sequence elements from a plurality of genomic region sequences to produce a plurality of genomic seed sequences;
(b) searching a plurality of target gene sequences with a multiplex query comprising a plurality of genomic seed sequences;
(c) identifying a plurality of target gene sequences substantially aligning with a plurality of genomic seed sequences, and
(d) locating regions of substantial alignment of said identified plurality of target gene sequences within said plurality of genomic region sequences, said regions of substantial alignment identifying a plurality of gene sequences.

23. The method of claim 22, further comprising obtaining gene specific nucleic acid sequence extending information within adjacent genomic region sequences for said identified plurality of gene sequences.

24. The method of claim 23, further comprising the steps of:
(a) searching a plurality of nucleic acid target sequences with a multiplex query comprising a plurality of gene seed sequences;
(b) identifying a plurality of target sequences substantially aligning with a plurality of gene seed sequences;
(c) selecting a plurality of substantially aligned target sequences containing nucleic acid sequence extending information for a plurality of gene seed sequences, and
(d) repeating steps (a) through (c) using said selected plurality of substantially aligned target sequences as a plurality of gene seed sequences.

25. The method of claim 24, further comprising repeating step (d) one or more times.

26. The method of claim 25, further comprising repeating said step (d) until identification of nucleic acid sequence extending information for said plurality of gene seed sequences is exhausted.

27. The method of claim 24, further comprising the step of clustering the selected plurality of substantially aligned target sequences containing nucleic acid sequence extending information to obtain a plurality of consensus nucleic acid target sequences.

28. The method of claim 27, further comprising aligning one or more of said plurality of consensus nucleic acid target sequences with one or more of the plurality of gene seed sequences to produce one or more extended gene seed sequences.

29. The method of claim 22, further comprising clustering said identified plurality of target gene sequences to obtain a plurality of consensus target gene sequences.

30. The method of claim 29, further comprising locating the regions of substantial alignment of said plurality of consensus target gene sequences within said plurality of genomic region sequences, said regions of substantial alignment identifying a plurality of gene sequences.

31. The method of claim 30, further comprising obtaining gene specific nucleic acid sequence extending information within adjacent genomic region sequences for said identified plurality of gene sequences.

32. The method of claim 31, further comprising the steps of:
(a) searching a plurality of nucleic acid target sequences with a multiplex query comprising a plurality of gene seed sequences;
(b) identifying a plurality of target sequences substantially aligning with a plurality of gene seed sequences;
(c) selecting a plurality of substantially aligned target sequences containing nucleic acid sequence extending information for a plurality of gene seed sequences, and
(d) repeating steps (a) through (c) using said selected plurality of substantially aligned target sequences as a plurality of gene seed sequences.

33. The method of claim 32, further comprising repeating step (d) one or more times.

34. The method of claim 33, further comprising repeating said step (d) until identification of nucleic acid sequence extending information for said plurality of gene seed sequences is exhausted.

35. The method of claim 32, further comprising selecting substantially aligned target sequences containing unidirectional nucleic acid sequence extending information.

36. The method of claim 32, further comprising selecting substantially aligned target sequences containing bidirectional nucleic acid sequence extending information.

37. The method of claim 32, further comprising identifying target sequences substantially aligning with about 90 base pairs (bp) or more of nucleic acid seed sequence.

38. The method of claim 32, further comprising selecting substantially aligned target sequences having about 40 bases (b) or more of nucleic acid sequence extending information.

39. The method of claim 32, further comprising pruning superfluous nucleic acid sequence information from said plurality of gene seed sequences or nucleic acid target sequences.

40. The method of claim 39, wherein said pruning is selected from the group of filtering, removing and masking of sequence information.

41. The method of claim 39, wherein said superfluous nucleic acid sequence information further comprises substantially abundant target sequences.

42. The method of claim 41, wherein said substantially abundant target sequences comprise about 500 or more substantial alignments with a seed sequence.

43. The method of claim 39, wherein said superfluous nucleic acid sequence information further comprises substantially overabundant members within a target sequence cluster.

44. The method of claim 43, wherein said target sequence clusters comprise greater than about 12,000 or more members.

45. The method of claim 39, wherein said superfluous nucleic acid sequence information further comprises internal or terminal sequence information.

46. The method of claim 39, wherein said pruning results in bidirectional or unidirectional identification of nucleic acid sequence extending information.

47. The method of claim 32, wherein said plurality of target sequences are selected from the group consisting of expressed sequence tags (ESTs), cDNA and genomic DNA, or fragments thereof.

48. The method of claim 32, wherein said plurality of gene seed sequences are selected from the group consisting of expressed sequence tags (ESTs), cDNA and genomic DNA, or fragments thereof.

49. The method of claim 32, wherein said multiplex query further comprises a concatenated plurality of gene seed sequences.

50. The method of claim 49, further comprising deconvoluting said identified plurality of target sequences into component nucleic acid target sequences.

51. The method of claim 32, further comprising the step of clustering the selected plurality of substantially aligned target sequences containing nucleic acid sequence extending information to obtain a plurality of consensus nucleic acid target sequence.

52. The method of claim 51, further comprising aligning one or more of said plurality of consensus nucleic acid target sequences with one or more of said plurality of gene seed sequences to produce one or more extended nucleic acid seed sequence.

53. The method of claims 22, 24 or 32, further comprising identifying within said gene sequences or gene seed sequences nucleic acid sequence elements selected from the group consisting of intron signals, poly-A regions, poly-A signals and structural motifs.

54. The method of claims 22, 24 or 32, further comprising annotating said gene sequences or genomic region sequences with nucleic acid sequence attributes.
